(19) United States
(12) Patent Application Publication
(10) Pub. No.: US 2012/0232622 A1
(43) Pub. Date: Sep. 13, 2012

(54) FAST HEATING HEAT PACKS WITH BINARY ACTION

(76) Inventors: Leo B. Kriksunov, Ithaca, NY (US); David W. Wynn, Huntington Valley, PA (US)

(21) Appl. No.: 13/414,026

(22) Filed: Mar. 7, 2012

Related U.S. Application Data
(60) Provisional application No. 61/451,324, filed on Mar. 10, 2011.

(51) Int. Cl. A61F 7/08

(52) U.S. Cl. 607/114

(57) ABSTRACT

A thermal treatment device having at least one fast-acting heat pack and at least one long lasting heat pack is disclosed.

This application claims the benefit of U.S. provisional 61/451,324 filed Mar. 10, 2011, the complete disclosure of which is hereby incorporated herein by reference for all purposes.

### FIELD OF THE INVENTION

The present invention is directed to a thermal treatment device. More particularly, the present invention relates to a thermal treatment device that has at least one fast-acting heat pack; and at least one long lasting heat pack.

### BACKGROUND OF THE INVENTION

For patients with aching muscles and sore joints, the application of heat can loosen stiff muscles, improve blood flow to the affected area, facilitate tissue repair, and create a feeling of relaxation. The application of heat to the skin has historically been used for pain relief of muscles and joints, as well as for the treatment of certain inflammatory conditions. The application of cold materials to the skin has also been used for similar treatments, especially for treating inflammatory responses such as joint inflammation.

Chemical formulations, such as iron powder formulations, that oxidize when exposed to air have been employed to generate heat. Electrical heating elements attached to a power source have also been employed.

U.S. Pat. No. 6,074,413 to Proctor & Gamble discloses a disposable thermal back wrap that contains one or more thermal packs.

U.S. Pat. No. 5,484,366 to Wilkinson discloses a belt that contains at least one packet to hold chemical gel packs. The gel packs may be heated or cooled to the desired temperature.

U.S. Pat. No. 5,605,144 discloses a garment that has at least one pouch for holding an air activated heat producing packet.

U.S. Application No. 20080200971 to Mycoal Products Corporation discloses a heating element that contains three or more segmented heating parts that contain an exothermic composition capable of heat generation upon contact with air that consists of a low temperature segment, an intermediate temperature segment and a high temperature segment.

U.S. Application No. 20080140165 to Kimberly-Clark Worldwide, Inc. discloses an article for delivering a heating sensation that includes a skin facing surface, a plurality of warm potions disposed on the skin facing surface and a plurality of cool portions disposed on the skin facing surface, wherein the warm potions and the cool portions are disposed in an alternating pattern.

U.S. Pat. No. 7,871,427 to Carewave, Inc. discloses a heating device that comprises a control unit that permits a ramp-up phase, a steady heat phase, a ramp-down phase and a soak heat phase, wherein the temperature differential between the steady heat phase and the soak heat phase is less than 5° C., wherein the soak heat phase is the lowest temperature in the heat cycle and wherein the soak heat phase is above normothermia. U.S. Pat. No. 7,841,427 also discloses a method of using heat to reduce accommodation of thermal nerve receptors on the skin of a subject that comprises: (i) providing a heating element on skin of a user; (ii) increasing the temperature of the heating element to provide a first temperature ramp-up period; (iii) holding the temperature of the heating element at a predetermined first predetermined therapeutic level for at least thirty seconds; (iv) decreasing the temperature of a heating device during a ramp-down period; and (v) holding the temperature of the heating device at a second predetermined soak level, wherein the soak level temperature is above a normothermic temperature.

EP 0330472 to Thermapeutics, Inc. discloses an apparatus for producing a series of temperature patterns for reducing pain. The temperatures are produced by means of thermoelectric elements and are in the range of between 19° C. and 44° C. A change in the temperature gradient takes place at most once per minute.

There is a need in the art for improved thermal treatment devices.

### SUMMARY OF THE INVENTION

Currently marketed air-activated heat packs heat up slowly, within about 30 minutes or longer. There is a consumer need to provide faster heat-up of the heat pack to initiate treatment sooner. Preferably, the heating should be within a few minutes after opening the pack. At the same time the pack should operate for at least about 8 hours or longer. At the same time, the pack should reach only safe temperatures so as to not to burn the user.

The thermal treatment device is convenient in that different compositions may be placed into different portions of the thermal treatment device, resulting in a pack which provides a hotter portion for a brief burst of time and a warmer portion for a longer portion of time. Typically, air activated heating device compositions are limited, in that the more rapidly the composition oxidizes upon exposure to air, the more rapidly it is extinguished. If a composition is extinguished too rapidly, it cannot provide long-term pain relief. In previous thermal treatment devices, manufacturers have had to compromise between the intensity of the heat and the duration of the heat delivery. It is advantageous to users to have a device that provides an intense heating portion at the beginning of use, followed by a longer warming device. This can be provided using the device of the present invention.

### BRIEF DESCRIPTION OF THE DRAWINGS

FIG. 1 is a graph showing a prophetic example of the use of the thermal treatment device of the present invention, wherein the temperature inside a fast-acting heat pack and the temperature inside the long-lasting heat pack are shown as a function of time, with zero time being the opening of the thermal treatment device, i.e., the opening of the air-impermeable packaging of the thermal treatment device. In another embodiment, one face or side of the thermal treatment device has a backing which can be “opened” by being removed, which allows for the ingress of air and subsequent exothermic reaction.

FIGS. 2A and 2B depict (a) a side view and (b) a top view of an embodiment of a thermal treatment device of the present invention, wherein the thermal treatment device contains at least two heat packs, i.e., at least one fast-acting heat pack and at least one long-lasting heat pack, wherein the fast-acting heat pack is disposed on the long-lasting heat pack.

FIGS. 2C and 2D depict (a) a side view and (b) a top view of an embodiment of a thermal treatment device of the present invention, wherein the thermal treatment device contains at least two heat packs, i.e., at least one fast-acting heat pack and at least one long-lasting heat pack, and an insulator, wherein the fast-acting heat pack is disposed on the insulator.

FIGS. 3A and 3B depict (a) a side view and (b) a top view of an embodiment of a thermal treatment device of the present invention, wherein the thermal treatment device contains at least two heat packs, i.e., at least one fast-acting heat pack and at least one long-lasting heat pack, a pocket for the fast-acting heat pack, a pocket for the long-lasting heat pack and a belt.

### DETAILED DESCRIPTION OF THE INVENTION

The following detailed description references the accompanying drawings that show some example aspects of the disclosure. These example aspects are described in sufficient detail to enable those skilled in the art to practice the disclosure. It is to be understood that other aspects may be utilized, or structural changes made, such that the detailed description should not be considered as limiting the scope of the claims.

Certain heat packs can heat up very quickly. This is achieved by using an appropriate heating mixture composition (e.g., iron powder, salts, water, active carbon and filler), particle size (e.g., finer particles have higher surface area and accelerate the reaction), and air permeability of the pouch (e.g., high air permeability results in faster heating). However, fast-acting heat packs last only for a short period of time, such as, for example, 0.5 hours, 1 hour, 3 hours. Also, fast-acting heat packs can reach very high temperatures which then can result in burns. Some of such fast-acting heat packs are used to heat a food ration for a soldier in the field. For example, U.S. Pat. No. 5,205,277 to Chao-Tsung discloses a self-heating container which employs three heat packs. The first heat pack contains calcium oxide and is the main component for producing heat. The second (medium) temperature heat pack contains an exothermic liquid composed of NaCl, acetic acid, and water. The third (high) heat pack contains an exothermic liquid composed of the same components as the medium heat pack, except in different ratios. The liquids in the medium and high heat packs are used to react with the calcium oxide, thereby releasing the heat of hydration to heat food.

U.S. Pat. No. 3,976,049 to Yamashita et al. discloses exothermic compositions consisting of iron powder, a chloride or sulfate, active carbon and water. U.S. Pat. No. 5,046,479 to Usui et al. discloses similar exothermic compositions based on iron powder.

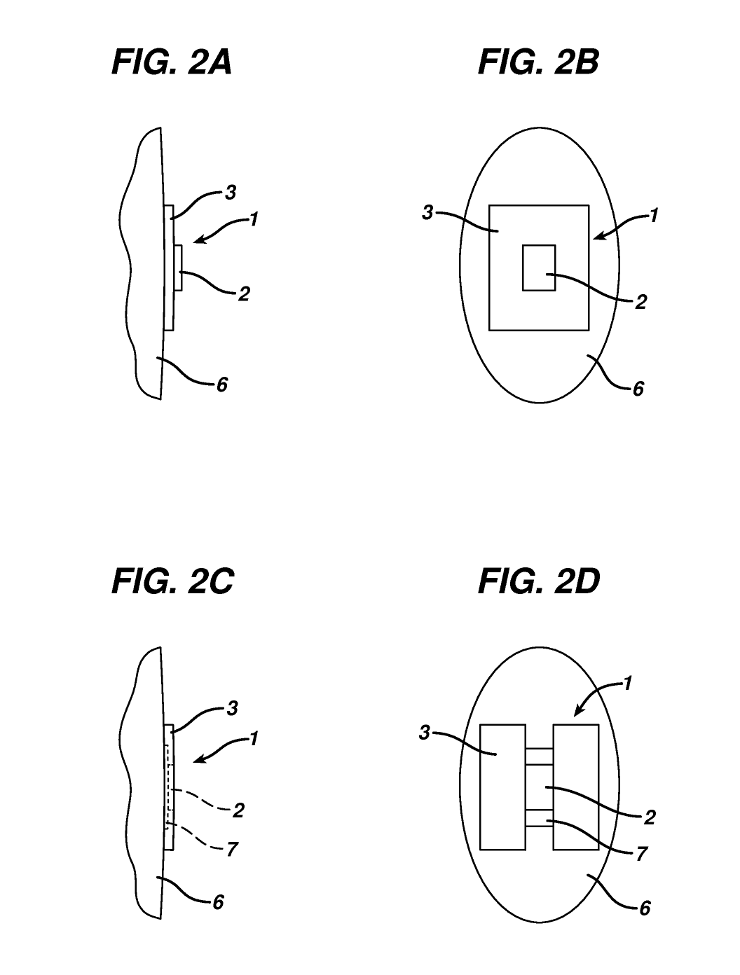

According to the present invention, the thermal treatment device 1 comprises at least two heat packs, i.e., at least one fast-acting heat pack 2 and at least one long-lasting heat pack 3, schematically shown in FIGS. 2-4. Both packs can be substantially flat with a thickness of about 1 mm to about 20 mm, more preferably from about 3 mm to about 10 mm. In one embodiment, the fast-acting heat pack 2 is smaller than the long-lasting heat pack 3.

According to an embodiment, the fast acting heat pack 2 is only providing heat for a brief period of time at the beginning of the treatment. Accordingly, the fast acting heat pack 2 may be comparatively smaller as the quantity of heat-generating material needed may also be smaller. Furthermore, due to the higher amount of heat generated by the fast acting pack, and the need to dissipate the higher amount of heat without causing heat injury, the fast acting heat pack may be smaller.

The fast-acting heat pack 2 is of usual construction, comprising an air-permeable pouch 4 filled with an air-activated heat mixture 5 except that (i) the air-activated heat mixture 5 has a specially formulated mixture to heat up faster (including, e.g., different concentrations of additives, additional components, such as salts or more active metals such as aluminum, and/or finer particle size of some of ingredients); (ii) the air-permeable pouch 4 has higher air permeability; or (iii) both (i) and (ii). The fast-acting heat pack 2, which heats up to its maximum temperature in about 5 to about 15 minutes, is capable of providing heat for up to about 15 minutes, up to about 30 minutes, up to about 60 minutes, or up to about 2 hours. Typically, the heat generating composition comprises from about 30% to about 80% iron powder; from about 3% to about 25% activated carbon, non-activated carbon, and mixtures thereof; from about 0.5% to about 10% metal salt; and from about 1% to about 40% water. For faster action, the composition may further include from about 1% to about 20% reactive metal powder such as aluminum or magnesium, or may further include finer iron powder.

The long-lasting heat pack 3 is of usual construction, comprising an air-permeable pouch 4 filled with an air-activated heat mixture 5, wherein the air-activated heat mixture 5, the air-permeable pouch 4; or (iii) both 4, 5 differ from that for the fast-acting heat pack 2 permitting the long lasting heat pack 3 to provide heat for up to about 8 hours, or up to about 12 hours, or up to about 24 hours.

In one embodiment the mean particle size of the air-activated heat mixture in the fast acting heat pack is substantially smaller than the mean particle size of the air-activated heat mixture in the long lasting heat pack, i.e., at least about 10% smaller, e.g., at least about 30% smaller, e.g., at least about 50% smaller, resulting in higher temperature and faster heating.

In one embodiment, the air permeability of the pouch of the fast acting heat pack is substantially higher than the air permeability of the pouch of the long lasting heat pack, i.e., at least about 10% higher, e.g., at least about 30% higher, e.g., at least about 50% higher, resulting in higher temperature and faster heating.

In one embodiment the heat generating composition of the fast acting heat pack contains more salt, more water, more fine particles, or additives of reactive metals such as aluminum or magnesium resulting in higher temperature and faster heating.

In order to avoid burning the user 6, the fast-acting heat pack 2 is not placed in direct contact with the user 6. Referring to FIGS. 2A and 2B, in one embodiment, the fast-acting heat pack 2 is placed behind the long-lasting heat pack 3. In this case the long-lasting heat pack 3 also serves as an insulator as well as a heat distributor between the user's body 6 and the fast-acting heat pack 2, preventing burns. Referring to FIGS. 2C and 2D, in another embodiment, the fast-acting heat pack 2 is placed alongside of the long-lasting heat pack 3, or between two long-lasting heat packs 3. According to one embodiment a thermal insulator 7 is placed between the user's body 6 and the fast-acting heat pack 2, or between the user's body 6 and the area on the long-lasting heat pack 3 behind which the fast-acting heat pack 2 is located.

The fast-acting heat pack 2 and the long-lasting heat pack 3 can both be part of the same multi-compartment pouch filled with different mixtures or having different air permeability. The fast-acting heat pack 2 and the long-lasting heat pack 3 can both be separate packs joined together by adhesive, by staples, by stitching, or by other means (not shown).

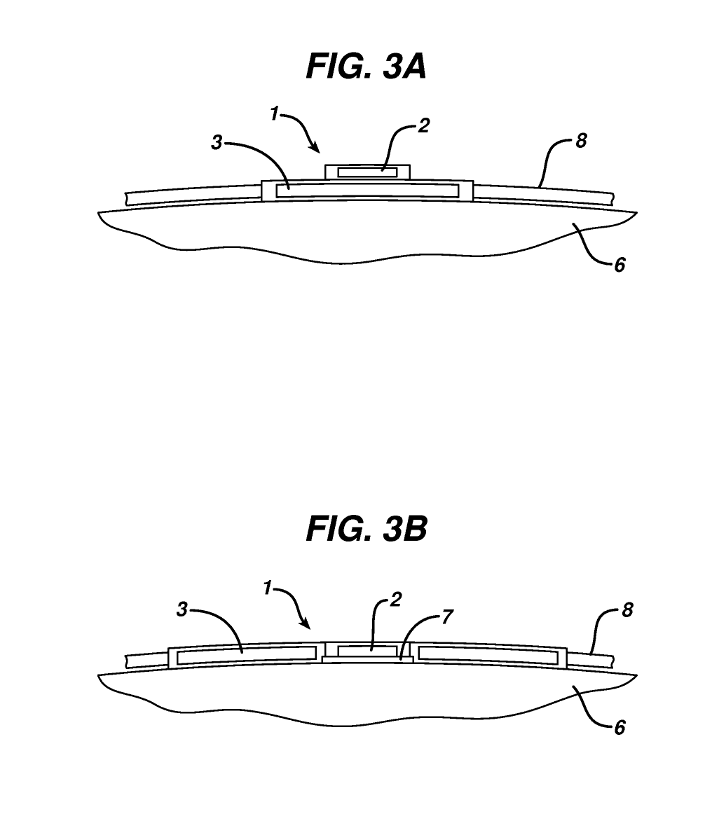

In all of the above embodiments, the thermal treatment device 1 comprising at least one fast-acting heat pack 2 and at least one long-lasting heat pack 3 can be worn on the user's body 6 via attachment with adhesive to the skin or to the clothing, by being inserted into a belt, sleeve, or wrap, or by securing with straps, having Velcro attachments, adhesive, or other means to attach to the body, clothing, or to another strap. Referring to FIGS. 3A and 3B, the fast-acting heat pack 2 and the long acting heat pack are worn on the user's body 6 using a belt 8.

In another embodiment the fast-acting heat pack 2 and the long-lasting heat pack 3 can both be discrete separate packs inserted into a wearable belt, wrap, or sleeve, with corresponding pockets provided for each pack, preferably size matching pockets to ensure placement of a correct pack in a correct pocket (not shown in the Figures). In one embodiment, the fast-acting heat pack 2 is inserted into a pocket behind another pocket for the long-lasting heat pack 3 (the pockets not shown in the Figures). In another embodiment, the fast-acting heat pack 2 is inserted into a pocket alongside the long-lasting heat pack 3, with an insulator 7 disposed between the user's body 6 and the pocket containing the fast-acting heat pack 2 (the pocket not shown in the Figures).

In one embodiment, the fast acting heat pack can be attached to a long lasting heat pack which heats at a lower temperature. In one embodiment, there is a backing which activates the heat packs upon removal. In one embodiment the fast acting heat pack and the long lasting heat pack have the same backing. In one embodiment, the fast acting heat pack has a first backing and the long lasting heat pack has a second backing.

In all embodiments, the temperature difference between the user's skin surface and the thermal treatment device 1 should be about 5-30° C., depending on the regulatory requirements for a particular product, thermal treatment device positioning on the user's body 6, configuration of the device and use time.

In all embodiments, the maximum temperature on the skin under the heat pack is not more than about 5-15° C. higher than the normal body temperature of 36-37° C. In all embodiments, the maximum temperature reached by the fast acting heat pack is at least about 5-20° C. higher than the steady-state temperature reached by the long lasting heat pack.

In all embodiments the maximum temperature of the fast acting heat pack is achieved before the long lasting heat pack achieves the steady-state temperature.

In operation, opening the thermal treatment device 1 results in rapid heating of the fast-acting heat pack 2, providing initial therapeutic heat treatment. While the long-lasting heat pack 3 heats up, the fast-acting heat pack 2 provides heat, and then starts to cool down. When the long-lasting heat pack 3 finally reaches the steady-state temperature, the fast-acting heat pack 2 cools and does not heat up any more having exhausted one or more reactive ingredients, for example water or metal powder.

In one embodiment, the long lasting heat pack circumvents the fast acting heat pack. In one embodiment, the fast acting heat pack rests on top of one face of the long lasting heat pack. In one embodiment, the fast acting heat pack is attached to the long lasting heat pack through a fastening means. In one embodiment, there exists a space between the fast acting heat pack and the long lasting heat pack which does not heat or is substantially free of a heating composition. This space may contain a thermally insulative portion which does not allow for the heat from one heat pack to be transferred to the other heat pack. This insulative portion may be made of materials including, but not limited to, foams, polystyrenes, plastics, silicone, silicone beads, and layered fabrics.

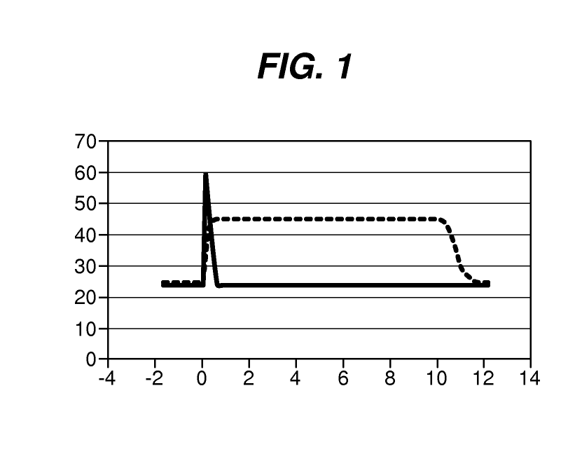

Referring to FIG. 1, a graph showing results of a prophetic example, wherein the temperature in degrees Celsius inside the fast-acting heat pack 2 and the temperature inside the long-lasting heat pack 3 are shown as a function of time in hours, with zero time being at the opening of the thermal treatment device.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

1. A thermal treatment device comprising:
at least one fast-acting heat pack; and
at least one long lasting heat pack.

2. The thermal treatment device of claim 1, wherein the fast-acting heat pack is disposed on the long-lasting heat pack.

3. The thermal treatment device of claim 1, wherein the fast-acting heat pack is disposed between two long lasting heat packs.

4. The thermal treatment device of claim 1, further comprising:
at least one pocket for the at least one fast-acting heat pack; and/or
at least one pocket for the at least one long-lasting heat pack.

5. The thermal treatment device of claim 1, further comprising a first thermal composition and a second thermal composition, wherein said first thermal composition is contained within said at least one fast-acting heat pack and said second thermal composition is contained within said at least one long lasting heat pack.

6. The thermal treatment device of claim 5, wherein the first thermal composition in said fast acting heat pack emits heat from about 1 to about 30° C. above the skin surface temperature of a user.

7. The thermal treatment device of claim 5, wherein said second thermal composition in said long lasting heat pack emits heat from about 1 to about 20° C. above the skin surface temperature of a user.

8. The thermal treatment device of claim 5, wherein said first thermal composition in said fast-acting heat pack emits heat up to about 30° C. above the skin surface temperature of a user within 15 minutes from start of use.

9. The thermal treatment device of claim 5, wherein said second thermal composition in said long acting heat pack emits heat up to about 20° C. above the skin surface temperature of a user within about one hour from start of use.

10. The thermal treatment device of claim 5, wherein a maximum temperature reached by the fast-acting heat pack is at least about 10° C. higher than a steady-state temperature reached by the long acting heat pack.

11. The thermal treatment device of claim 10,
wherein the fast-acting heat pack is adapted to reach the maximum temperature within a first time after opening the thermal treatment device,
wherein the long acting heat pack is adapted to reach the steady state temperature within a second time after opening the thermal treatment device,
and wherein the maximum temperature is higher than the steady state temperature by at least about 5° C.

12. The thermal treatment device of claim 11, wherein said first time is less than 30 minutes, said second time is less than 60 minutes, and wherein said first time is less than said second time.

13. The thermal treatment device of claim 5, wherein the first thermal composition and the second thermal composition are the same, and wherein the fast-acting heat pack has a first impermeable enclosure and the long lasting heat pack has a second air permeable enclosure, and wherein permeability of said first air permeable enclosure is higher than permeability of said second air permeable enclosure.

14. The thermal treatment device of claim 5, wherein the thermal composition comprises iron powder.

15. A method of treatment comprising:
exposing a user's skin to a thermal treatment device, comprising:
at least one fast-acting heat pack; and
at least one long lasting heat pack.

16. The thermal treatment device of claim 1, wherein a thermally insulating material is disposed between said fast-acting heat pack and a body of a user of said thermal treatment device, said thermally insulating material adapted to prevent a thermal injury to the user.
